(19) United States
(12) Patent Application Publication
(10) Pub. No.: US 2006/0281991 A1
(43) Pub. Date: Dec. 14, 2006

(54) Fiducial marker holder system for surgery

(76) Inventors: J. Michael Fitzpatrick, Nashville, TN (US); Robert F. Labadie, Nashville, TN (US); Robert L. Galloway Jr., Nashville, TN (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(21) Appl. No.: 10/526,754

(22) PCT Filed: Jan. 21, 2004
(86) PCT No.: PCT/US04/01441

§ 371 Date: Mar. 4, 2005

Related U.S. Application Data
(60) Provisional application No. 60/469,229, filed on May 9, 2003.

(51) Int. Cl. A61B 5/05

(52) U.S. Cl. 600/426

(57) ABSTRACT

A fiducial marker holder apparatus (20, 120, 220) includes a maxillary holding device (50) and an open-ended frame (30, 130, 230). The maxillary holding device (50) is configured to be temporarily secured to only a maxillary-region of a patient. The open-ended frame (30, 130, 230) has first and second arms (32, 132, 232, 33, 133, 233) and is configured to be removably attached to the maxillary holding device (50). The first arm (32, 132, 232) has at least one marker attachment point (48a-48h, 148a-148n, 248a-248l) that receives fiducial markers (48) and the second arm (33, 133, 233) has a plurality of marker attachment points (48a-48h, 148a-148n, 248a-248l) that receive fiducial markers (48). At least two of the marker attachment points (48a-48h, 148a-148n, 248a-248l) of the second arm (33, 133, 233) are configured to receive fiducial markers (48) in different orientations with respect to the open-ended frame (30, 130, 230) and each other (48a-48h, 148a-148n, 248a-248l).

### CROSS-REFERENCE TO RELATED APPLICATIONS

This application claims the benefit of U.S. Provisional Patent Application No. 60/469,229, filed May 9, 2003, entitled “Fiducial Marker Holder System For Surgery.”

### BACKGROUND OF THE INVENTION

The present invention generally relates to markers and marker systems for frameless stereotaxis imaging surgical procedures, and more particularly, to a fiducial marker holder system for otologic image-guided surgery adapted to increase registration accuracy.

Systems that assist surgeons in their navigation through anatomy are called “image-guidance” systems for performing image-guided surgery (IGS). Stereotaxic systems or stereotaxy is a method in neurosurgery and neurological research for locating points within the brain using an external, three-dimensional frame of reference usually based on the Cartesian coordinate system. Typical systems provide assistance by displaying the position of a surgical probe in the operating room relative to the anatomy of the patient (i.e., stereotaxis) via an image that was acquired pre-operatively. IGS consists of real-time display of current location on images during surgery. Analogous to Global Positioning Systems, IGS systems provide intraoperative navigational information which has been shown to be particularly useful in complex cases such as those where anatomy has been distorted by disease and/or in “limited-access” surgery where small incisions are used. IGS systems are currently available for neurosurgery, sinus surgery, and orthopedic surgery.

Imaging systems such as computed tomographic (CT) x-ray imagers, positron emission tomographic (PET) scanners, single photon emission computed tomography (SPECT) scanners and nuclear magnetic resonance imaging (MRI) machines provide the ability to improve visualization of the anatomical structure of the human body without surgery or other invasive techniques. A patient can be scanned by one or more of these imaging systems, and the patient's anatomical structure can be reproduced in a form for evaluation by a trained doctor. One problem associated with these scanning techniques concerns the accurate selection and comparison of views of identical areas in images that have been obtained by imagers at different times or by images obtained essentially at the same time using different image modalities. In order to relate information in an image of the anatomy to the anatomy itself, it is necessary to establish a one-to-one mapping between points in the image and points of anatomy which is referred to as registering “image space” to “physical space.” When registering two arbitrarily oriented three dimensional image, it is desirable to align the coordinate systems of the two images such that any given point in the scanned anatomy is assigned identical addresses in both images. The calculation of the rigid body transformation necessary to register the two coordinate systems requires knowledge of the coordinate vectors of at least three points in the two systems. Such points are called “fiducial points” or “fiducials,” and the fiducials used are the geometric centers of markers, which are called “fiducial markers.” These fiducials are used to correlate “image space” to “physical space” and to correlate one image space to another image space. The fiducial markers provide a constant frame of reference visible in a given imaging mode to make registration possible.

The general concepts for using fiducial markers to obtain registration of image data across time and of image-guided surgery are set forth in U.S. Pat. Nos. 4,991,579; 5,142,930; 5,230,338; 5,394,875; 5,551,429; 5,916,164; 6,106,464; 6,333,971 B2; and 6,584,339 B2; the contents of which are incorporated by reference herein.

Typical landmarks used for registration are anatomic points or fiducial markers attached to the skin or implanted in bone. Fiducial markers are localizing devices on the patient which can be easily identified in images. The standard for fiducial markers has been a rigid frame which is screwed directly into the patient's skull under local anesthesia. Registration involves identifying the fiducial markers on both the patient and the images and then linking or superimposing the image onto the patient. Proper alignment of the images with the patient is critical and technologically difficult. Registration creates a transformation matrix which allows a direct mapping of the patient's current anatomy to the corresponding preoperative radiographic images. Once registration has taken place, using an electronically visible probe (e.g., an infrared optical system or electromagnetic system) to detect fiducial marker locations in operative space which is registered to the same markers in radiographic space, the probe can be used as a pointer to identify surgical anatomy on a CT image or MRI image.

To limit error in IGS systems, registration is preferably performed by a preoperative scan to a surgical field of interest in the anesthetized patient. Registration landmarks (anatomic landmarks or fiducial placement) need to be immobile relative to the anatomy and arranged such that they surround the surgical field of interest. While multiple anatomic landmarks would initially appear useful, soft tissue (e.g. skin and muscle) relaxes and distorts under general anesthesia making boney landmarks necessary for accurate registration. A solution has been to implant markers into bone. This is routinely used in neurosurgery where screws are placed directly into the cranium prior to preoperative radiographic imaging and these screws serve as landmarks for registration. While accuracy with such systems is impressive, it involves the invasive placement of bone screws with small, but real, risk of infection and cosmetic deformity. Another solution is to use skin markers or skin contours. But, such systems have shown decreased accuracy which is unacceptable in otologic applications. Generally, skin affixed markers produce accuracies around 1.5 millimeters (mm) and laser skin contouring results in accuracies around 2 mm.

One multi-modal imaging marker system (i.e., fiducial marker system) is Acustar™ commercially available from Z-KAT, Inc., Hollywood, Fla. The accuracy of the Acustar™ system is derived from its marker design and the rigid placement of the markers directly on the skull. Anchoring fiducial markers by drilling directly into the skull may be acceptable in neurosurgical procedures where life-threatening tumors are often encountered. However, in ear surgery (i.e., otologic surgery), such invasive fiducial markers are undesirable given the less-severe nature of the disease and the general success in ear surgery without image-guidance.

In ear surgery (otologic surgery), the use of IGS systems has also been limited by technical difficulties in (1) achieving the submillimeter precision needed to avoid damaging adjacent structures including the facial nerve (which controls voluntary movement of the face), the inner ear (which allows hearing and balance control), major blood vessels from the brain to the body (the carotid artery and internal jugular vein), and the brain while (2) achieving this in a non-invasive fashion (i.e., not drilled into the skull). These difficulties relate both to fiducial marker placement and intraoperative registration.

One method for relating a pre-operative image to the anatomy itself is to non-invasively attach fiducial markers to the patient before imaging (i.e., surveying type markers that give fixed locations relative to the patient). By relating an internal structure to these markers geometrically in the pre-operative image, it is possible to determine its position during surgery, even if it is not visible. This idea has been used for decades in surgery, and one method that has been used for neurosurgery is to mount the markers to the upper mandible, using a device, sometimes called a “bite block,” that attaches to the teeth.

In order to maximize accuracy, fiducial markers may be mounted on a large frame that extends from the mouth to surround the area of the surgery, which in neurosurgery, is the brain. Mounting the markers on this large frame, instead of the relatively small bite block, permits their configuration to be larger. Larger configurations have been shown to provide, in general, greater registration accuracies. For example, U.S. Pat. No. 6,096,048 (“Howard, III et al.,”) discloses one such noninvasive, re-attachable skull-mounted fiducial marker system. A U-shaped frame detachably extends from a “bite-block” and is used to mount the markers. Several other related earlier patents include U.S. Pat. Nos. 5,676,673; 5,829,444; 5,803,089; 5,873,822; 5,967,980; and 6,175,756, which are all based on magnetic localization and all but one deal with a headset. In particular though, U.S. Pat. No. 6,175,756 (“Ferre et al.”) discloses a position tracking and imaging system for use in medical applications.

It is desirable to provide a fiducial marker holder system for otologic surgery adapted to increase registration accuracy. It is also desirable to provide a fiducial marker holder system used in combination with an upper maxillary clamping device (i.e., a mouthguard) to increase stability. Even further, it is desirable to provide a fiducial marker holder system and image-guided otologic surgical system having accuracies in the submillimeter range and which is non-invasive and non-obstructive.

### BRIEF SUMMARY OF THE INVENTION

The present invention comprises a fiducial marker holder apparatus including a maxillary holding device and an open-ended frame. The maxillary holding device is configured to be temporarily secured to only a maxillary-region of a patient. The open-ended frame has first and second arms, and the open-ended frame is configured to be removably attached to the maxillary holding device. The first arm has at least one marker attachment point that receives fiducial markers and the second arm has a plurality of marker attachment points that receive fiducial markers. At least two of the marker attachment points of the second arm are configured to receive fiducial markers in different orientations with respect to the open-ended frame and each other.

The present invention also comprises a method of performing image-guided surgery on a patient using a maxillary holding device, an open-ended frame, a plurality of fiducial markers, a reference emitter, a surgical probe/instrument and an image-guided surgical system. The method includes attaching the open-ended frame with the plurality of fiducial markers to a patient using the maxillary holding device; acquiring a preoperative scan of the patient and the fiducial marker holder apparatus with the plurality of fiducial markers; removing the maxillary holding device from the patient; making a surgical plan, by the surgeon, from the preoperative scan; calculating the position of any point in or on the patient relative to the frame; attaching the reference emitter to the frame; activating the tracking sensor which then begins tracking the reference emitter and the frame; calibrating the frame and the reference emitter, while the patient is being prepared for surgery, so that the position of the frame relative to the reference emitter is determined; calculating the position of the frame relative to the reference emitter; removing the frame with the plurality of fiducial markers from maxillary holding device; attaching the maxillary holding device with the reference emitter the to a patient using; calculating the position of any point in the intraoperative-imaged patient anatomy relative to the reference emitter; activating a surgical probe/instrument; tracking the reference emitter and the surgical probe/instrument simultaneously; calculating the position of the surgical instrument relative to the patient's anatomy; and using the image-guided surgical system to guide surgery.

The present invention further comprises a method of calibrating an image-guided surgical system that is used to perform image-guided surgery on a patient using a maxillary holding device, an open-ended frame, a plurality of fiducial markers, a reference emitter, a surgical probe/instrument and an image-guided surgical system having memory and a tracking sensor. The method includes attaching the open-ended with the plurality of fiducial markers to a patient using the maxillary holding device; acquiring a preoperative scan of the patient and the fiducial marker holder apparatus with the plurality of fiducial markers; removing the maxillary holding device from the patient; attaching the reference emitter to either the open-ended frame or the maxillary holding device; activating the tracking sensor which then begins tracking the reference emitter; activating the surgical probe/instrument; tracking the reference emitter and the surgical probe/instrument simultaneously and continuously calculating the position of the surgical probe/instrument relative to the reference emitter; and calibrating the open-ended frame with fiducial markers and the reference emitter by touching the surgical probe/instrument to each fiducial marker, so that the position of the open-ended frame relative to the reference emitter is determined and stored in the memory of the image-guided surgical system.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a perspective view of a first preferred embodiment of a fiducial marker holder apparatus for surgery attached to a maxillary holding device which is attached to the maxillary jaw and/or teeth of a human skull;

FIG. 2 is a side view of the fiducial marker holder apparatus of FIG. 1;

FIGS. 3A-3C are top plan views of the fiducial marker holder apparatus of FIG. 1;

FIG. 4 is a side view of a maxillary holding device which is attached to the maxillary jaw and/or teeth of a human skull;

FIG. 5 is a top plan view of the maxillary holding device of FIG. 4;

FIG. 6 is a front elevational view of the maxillary holding device of FIG. 4;

FIG. 7 is a perspective view of a second preferred embodiment of a fiducial marker holder apparatus for surgery attached to a maxillary holding device which is attached to the maxillary jaw and/or teeth of a human skull;

FIG. 8 is a side elevational view of the fiducial marker holder apparatus of FIG. 7;

FIG. 9 is a top plan view of the fiducial marker holder apparatus of FIG. 7;

FIG. 10 is a front elevational view of the fiducial marker holder apparatus of FIG. 7;

FIG. 11 is a greatly enlarged view of a fiducial marker attachment device;

FIG. 12 is a perspective view of a third preferred embodiment of a fiducial marker holder apparatus for surgery before being attached to a maxillary holding device which will be attached to the maxillary jaw and/or teeth of a human skull along with a reference emitter;

FIG. 13A is a front elevational view of the fiducial marker holder apparatus of FIG. 12 as configured for a preoperative scan;

FIG. 13B is a side elevational view of the fiducial marker holder apparatus of FIG. 13A;

FIG. 13C is a top plan view of the fiducial marker holder apparatus of FIG. 13A;

FIG. 14A is a front elevational view of the fiducial marker holder apparatus and maxillary holding device of FIG. 12 with the reference emitter attached and being configured for a calibration procedure;

FIG. 14B is a side elevational view of the fiducial marker holder apparatus of FIG. 14A;

FIG. 14C is a top plan view of the fiducial marker holder apparatus of FIG. 14A;

FIG. 15A is a front elevational view of the maxillary holding device of FIG. 12 with the reference emitter attached and being configured for a surgical procedure;

FIG. 15B is a side elevational view of the maxillary holding device and reference emitter of FIG. 15A;

FIG. 15C is a top plan view of the maxillary holding device and reference emitter of FIG. 15A;

FIG. 16 is a greatly enlarged view of a fiducial marker;

FIG. 17 is a rendering demonstrating the parameters of rigid-body motion;

FIG. 18 is a rendering demonstrating image registration, fiducial registration error (FRE), fiducial localization error (FLE), and target registration error (TRE);

FIG. 19 is an exploded view of a locking dental acrylic resin splint (LADS);

FIG. 20 is an enlarged bottom plan view of a LADS with setscrews for mounting to a patient's maxillary teeth;

FIG. 21 is a diagram depicting an exemplary image-guided surgical system that can be used with the preferred embodiments of the present invention; and

FIG. 22 is a diagram depicting an exemplary image-guided surgical system in an operating room (OR) environment with a reference emitter and a “patient.”

### DETAILED DESCRIPTION OF THE INVENTION

Certain terminology is used in the following description for convenience only and is not limiting. The words “right”, “left”. “lower”, and “upper” designate directions in the drawings to which reference is made. The words “inwardly” and “outwardly” refer to directions toward and away from, respectively, the geometric center of the object discussed and designated parts thereof. The terminology includes the words above specifically mentioned, derivatives thereof and words of similar import. Additionally, the word “a” as used in the claims and in the corresponding portions of the specification, means “one or more than one.” Further, as used herein, “maxillary teeth” are the teeth protruding from the upper jaw, and “maxilla” is the upper jaw or upper jaw bone. Furthermore, as used herein, “maxillary” or “maxillary-region” is used to pertain to either the upper jaw, the region encompassing the upper jaw with teeth protruding therefrom and/or simply the teeth protruding from the upper jaw (maxilla).

The preferred embodiments of the present invention are intended to be utilized during at least a preoperative imaging scan in order to provide points of reference within a scanned image for a surgeon and/or image-guided surgical control software and/or for image registration calculations. The preferred embodiments of the present invention may also be used at other stages of an overall image-guided surgical procedure including during equipment calibration and during actual surgery. As used herein, “image-guided surgical procedures” may include preoperative scans, preoperative probing procedures, preoperative calibration, surgical scans, surgical control, surgical procedures and post-operative scans, among other things. A typical image-guided surgical procedure includes performing a preoperative scan, making a surgical plan, performing any calibrations and then performing the actual surgical procedure directly by the surgeon or by an automatic or semi-automatic control system (not shown).

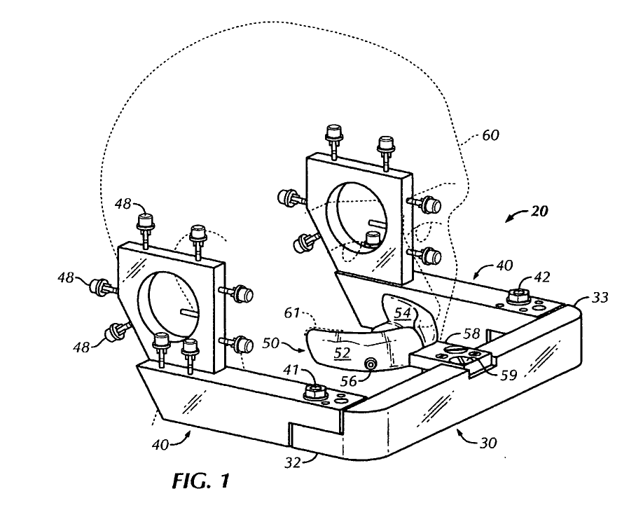

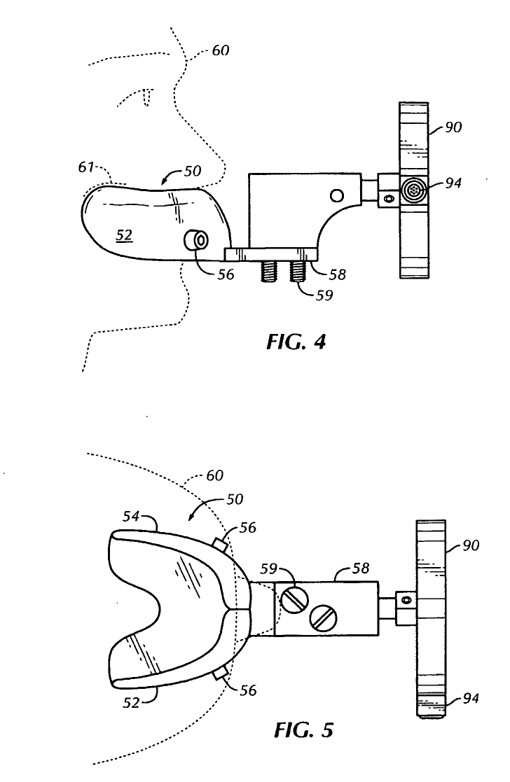

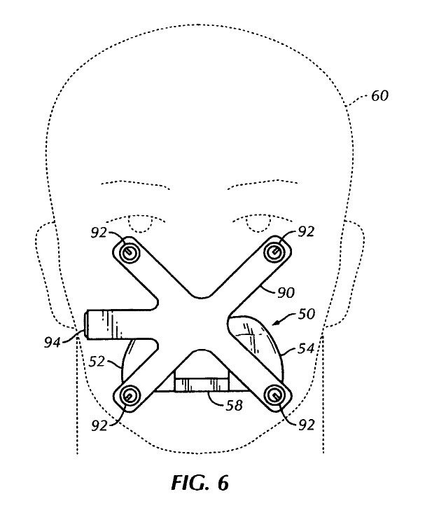

Referring to the drawings in detail, there is shown in FIGS. 1-2 and 3A-3C a first preferred embodiment of a fiducial marker holder apparatus for surgery 20 that is shown attached to a maxillary holding device 50 which is attached to the maxillary jaw and/or teeth 61 of a human skull 60. The fiducial marker holder apparatus 20 includes an open-ended frame 30 and a marker attachment device 40. The open-ended frame 30 is configured to be removably attached to the maxillary holding device 50 (shown in detail in FIGS. 4-6). As shown, the open-ended frame 30 is secured to a mounting plate 58 by means of setscrews 59, but any attachment mechanism may be utilized without departing from the broad inventive scope of the present invention.

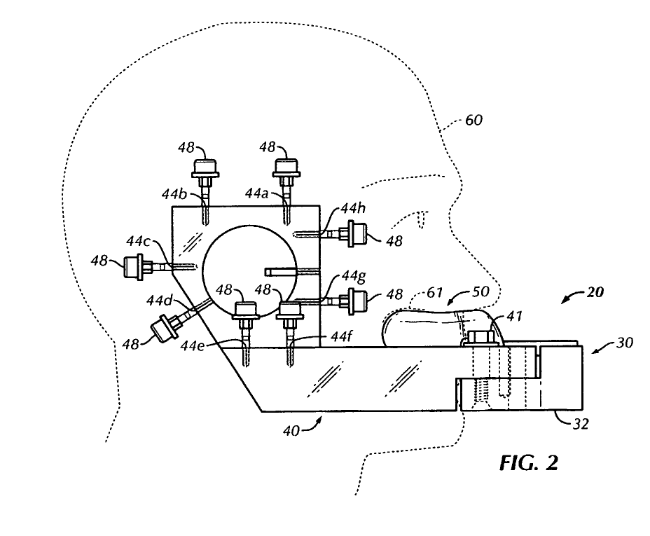

The marker attachment device 40 is disposed on a first end or arm 32 of the open-ended frame 30. Preferably, there is a marker attachment device 40 on both the first arm 32 and a second end or arm 33 of the open-ended frame 30. As best shown in FIG. 2, each marker attachment device 40 has a plurality of marker attachment points 44a-44h for fiducial markers 48. The fiducial markers 48 may be any type of fiducial marker compatible with magnetic resonant imaging (MRI), Computerized Axial Tomography (CAT scan) or other surgical imaging and/or guidance equipment as is known in the art. Each of the marker attachment points can be a threaded socket, a threaded post, a through-hole, a post, a socket, a detent, and the like as would be obvious to one skilled in the art. In effect, any attachment mechanism may be implemented for temporarily or permanently mounting fiducial markers 48 to the marker attachment device 40 and/or open-ended frame 30 without departing from the present invention. Preferably, the first arm 32 has at least one marker attachment point 48a-48h that receives fiducial markers 48 and the second arm 33 has a plurality of marker attachment points 48a-48h that receive fiducial markers 48, and at least two of the marker attachment points 48a-48h of the second arm 33 are configured to receive fiducial markers 48 in different orientations with respect to the open-ended frame 30 and each other 48a-48h. Each marker attachment device 40 has multiple, distinct geometric surfaces and the marker attachment points 48a-48h are disposed on two or more of the surfaces of the marker attachment device 40 thereby facilitating arranging the fiducial markers 48 in different orientations with respect to the open-ended frame 30 and some other fiducial markers 48. Thus, fiducial markers 48 on each of the arms 32, 33 can be arranged in two or more dimensions with respect to the arm 32, 33 to which they are attached.

FIG. 16 is a greatly enlarged view of a fiducial marker 48 which can be used with any and all of the preferred embodiments. While one particular type of fiducial marker 48 is shown, it should not be construed as limiting. The embodiments of the present invention may utilize any other suitable fiducial marker 48. Generally, there are fiducial markers 48 which are used for acquiring scans (i.e., that show up during a scan) and fiducial markers 48 used for calibration (i.e., that have a calibration point or divot). Thus, the fiducial markers 48 are localizable in preoperative scans and can either be localized physically or can be replaced by other fiducial markers 48 that can be localized physically. However, for simplicity, both varieties of fiducial markers 48 will be referred to as fiducial markers 48.

Since the embodiments of the present invention can be used during the imaging process (e.g., a preoperative scan) and during the surgical procedure itself, the only potential difference is the nature of the markers 48 that are used in the two circumstances. After imaging, but before the surgical procedure begins, the frame 30 having fiducial markers 48 attached thereto may be replaced by a second frame 30 having different fiducial markers 48 that can be tracked during the surgery. Alternatively, the fiducial markers 48 can be changed or replaced on the same frame 30. It is also possible for the fiducial markers 48 to be used during a preoperative scan, but not during the actual surgical procedure as will be described in detail hereinafter.

The open-ended frame 30 and the marker attachment devices 40 are formed of a material that is inert or innocuous to MRIs, CT scans and the like, such as a polymeric material or a non-metalic composite material. Preferably, the open-ended frame is formed of a rigid carbon-fiber which is selected for its high stiffness (rigidity) to weight ratio. But, the open-ended frame 30 and the marker attachment devices 40 may be formed of other materials without departing from the present invention.

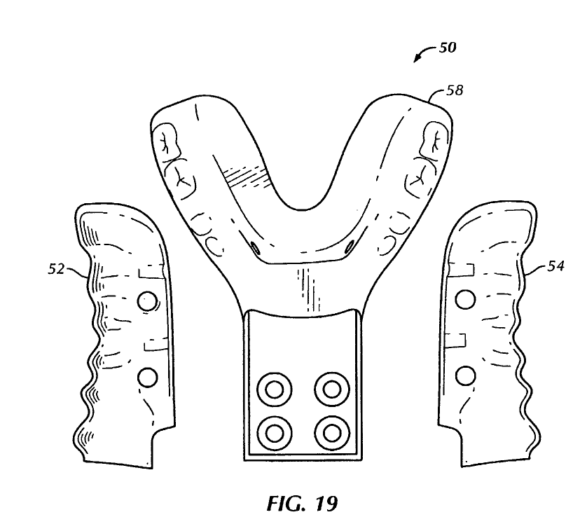

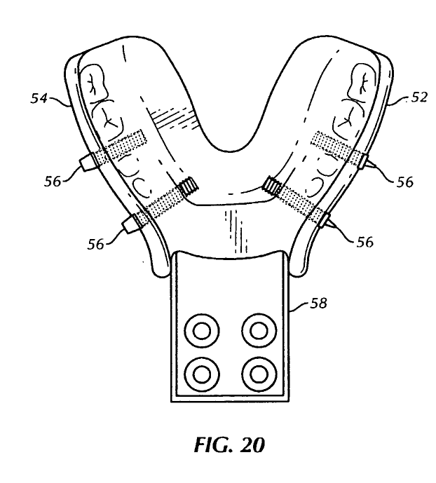

It is desirable to have a stable (nearly rigid) attachment to patient's anatomy such as by attachment to the upper or maxillary jaw and/or teeth provided by a holding device or “bite block.” The bite block can be of alternate designs, but preferably the maxillary holding device 50 (shown in detail in FIGS. 4-6) is similar to that designed by Dr. Michael Fenlon of Kings College, London. That maxillary holding device 50 has a first clamping part 52, a second clamping part 54 and a fixing tool or tools 56. The fixing tools 56 are configured to temporarily secure the first and second clamping parts 52, 54 to only a maxillary-region 61 of a patient. The fixing tools 56 may be setscrews, pins, nut/bolt combinations or the like which are suitable fore securely fixing the maxillary holding device 50 to the teeth and/or jaw bone. The maxillary holding device 50 has previously been tested by Dr. Fenlon and his colleagues in a neurosurgical application. This particular design for a maxillary holding device 50 attaches rigidly and securely to the teeth (not shown clearly) of the maxillary jaw-structure 61 without relying on pressure from the lower or mandibular jaw (not shown) to hold it in place. FIGS. 19-20 show a locking-acrylic dental splint (LADS) which forms the maxillary holding device 50. The maxillary holding device 50 (i.e., mouthpiece) includes the three major parts 52, 54, 58 shown in the exploded view of FIG. 19 as well as the fixing devices 56 shown in the assembled bottom plan view of FIG. 20. The geometry of typical teeth, being narrower at the insertion into the gums, ensures a tight, reliable fit. The maxillary holding device 50 is basically a modified dental bite-block that is custom molded for individual maxillary dental patterns. For fitting purposes, the maxillary holding device 50 is broken down into a central portion 58 which imprints the lingual and occlusal surfaces of the maxillary teeth as well as right and left pieces 52 and 54 which imprint the buccal surfaces and attach to the central portion 58 locking the maxillary holding device 50 onto the maxilla 61. An extension from the central piece 58 allows mounting of external hardware such as the fiducial frame 30, 130, 230 and/or the reference emitter 90. As mentioned above, any maxillary holding device which is configured to securely fix the maxillary teeth and/or jaw bone (maxilla) can be utilized without departing from the present invention.

The broad general concept of a marker-holding frame that attaches rigidly but releasably to a “bite block” is not novel per se. However, a significant difference provided by the present invention is that the open-ended frame 30 permits adjusting the placement and configuration of the marker attachment devices 40 and/or the fiducial markers 48 to optimize registration accuracy for the particular combination of procedures and/or for a particular patient's anatomy. By being able to concentrate fiducial markers 48 in the area of interest (i.e., the area surgical procedure will be performed) and by being able to configure the fiducial markers 48 in two-dimensions or more relative to each marker attachment device 40, the registration accuracy in the area of interest can be greatly improved.

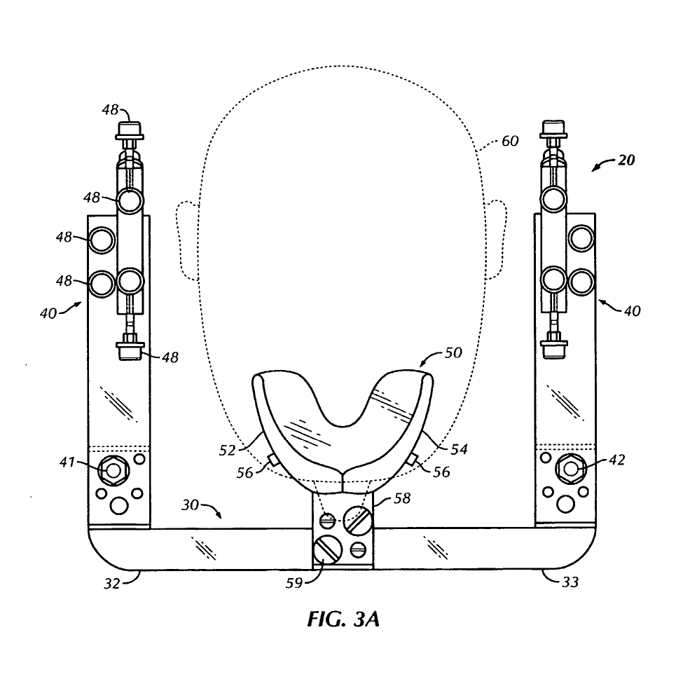

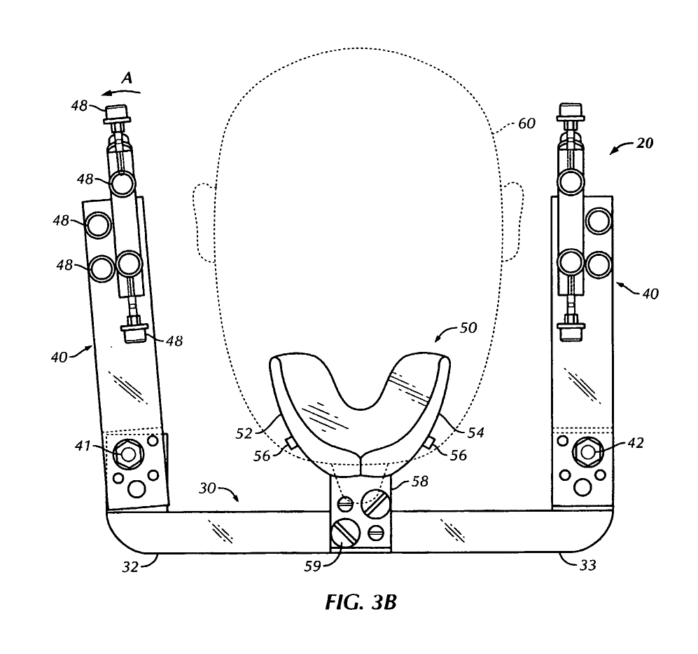

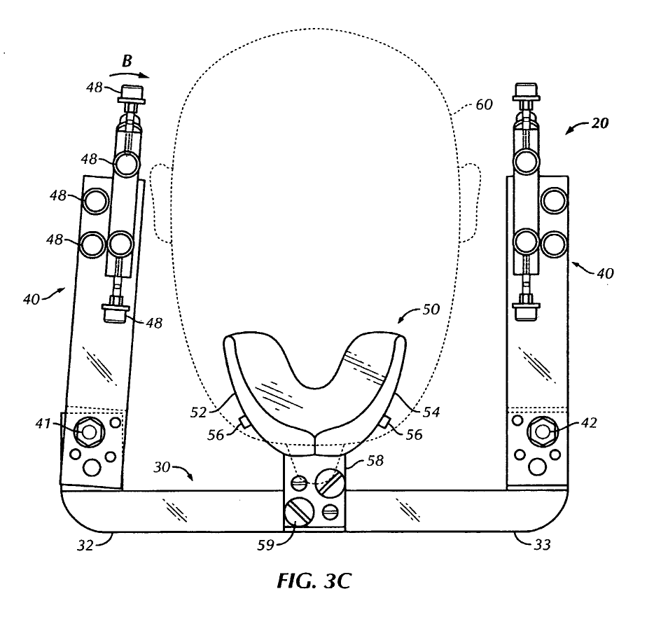

The marker attachment device 40 can be movably attached to the open-ended frame 30. As best seen in FIGS. 3A-3C, the distal ends 32 and 33 include attachment mechanisms 41 and 42 for rotatably attaching the marker attachment devices 40 thereto. The attachment mechanisms 41 and 42 shown are merely posts or set screws; however, other more complex movable attachment mechanisms 41, 42 may be utilized including meshing gears, ratcheting devices, more complex hinges and the like. The attachment mechanisms 41, 42 may be configured to be moveable in three degrees of freedom (e.g., pitch, yawl and roll) by way of a socket joint or the like. But, preferably, the attachment mechanisms 41, 42 are capable of being fixedly oriented, at least temporarily, in one of several positions. Thus, the fiducial marker attachment devices 40 are configured to be permanently or temporarily fixedly oriented in more than one position. Preferably, the attachment mechanisms 41, 42 are indexed or otherwise marked to permit repeatable placement in a particular position or configuration after reorienting the fiducial marker attachment devices 40 from another position or configuration in order to reduce human error. It is desirable that the repeatable placement be performed with a degree of precision.

In the first preferred embodiment, the fiducial marker attachment devices 40 are generally wide, flat, paddle-like, pieces which are intended to be positioned near the area of the ear. The fiducial marker attachment devices 40 preferably provide a two- or three-dimensional array of points (marker attachment points 44a-44h) on which fiducial markers 48 can be mounted. The fiducial marker attachment devices 40 may be other shapes and configurations such as curvilinear, square, circular, L-shaped, X-shaped and the like without departing from the invention. Since the fiducial marker attachment devices 40 are movably mounted to the open-ended frame 30 in such a way that permits them to be rotated and/or shifted, the fiducial marker attachment devices 40 each have two or more orientations and/or positions that may be selected relative to the open-ended frame 30 that joins it to the maxillary holding device 50. The purpose for such adjustability is to allow the configuration of the fiducial markers 48 to be moved to accommodate a patient's size or shape by being moved outwardly (the direction of arrow A in FIG. 3B) or by being moved inwardly (the direction of arrow B of FIG. 3C) and for being farther from or closer to the targeted area for purposes of altering the resolution in a particular region.

Human error is a concern in restoring fiducial marker attachment devices 40 to the precise or nearly precise position as they were during a respective preoperative scan at the point when the surgical procedure will take place. Further, not only is human error a concern in restoring fiducial marker attachment devices 40 to the precise or nearly precise position during a respective preoperative scan, but mechanical “play” or “slop” between the fiducial marker attachment devices 40 and the frame 30 can also inadvertently and undesirably introduce error. It is therefore contemplated that the fiducial marker attachment devices 40 can be permanently attached to the frame 30 to prevent or reduce the introduction of such mechanical error (e.g., integrally molded or machined into a singular part). For embodiments having the fiducial marker attachment devices 40 permanently fixedly attached to the frame 30 or integral with the frame 30, a variety of sizes and/or shapes of frames 30 and/or fiducial marker attachment devices 40 can be provided to accommodate a variety of patient sizes and shapes.

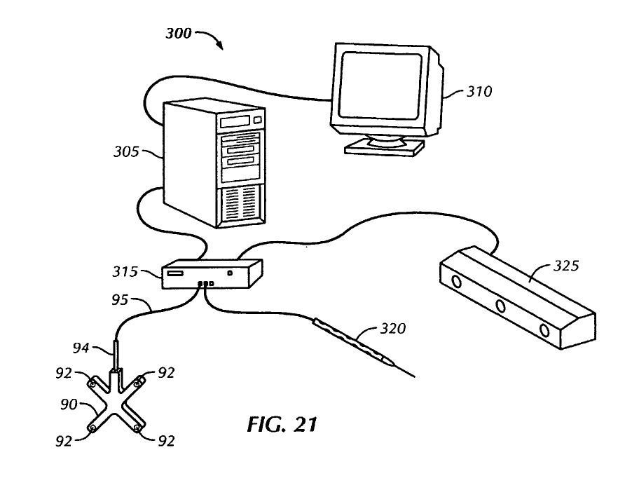

FIG. 21 shows an exemplary image-guided surgical system 300 that can be used with the preferred embodiments of the present invention. The image-guided surgical system 300 includes a computer (an image data processor) 305 with memory and a display monitor 310. A high-speed interface in the computer 305 communicates with a surgical probe/instrument 320 via control box 315, although the features of the control box 315 could be incorporated directly into the computer 305. Additional hardware for implementing the exemplary image-guided surgical system 300 include an optical tracking sensor 325 and a reference emitter 90. The surgical probe/instrument 320 may be an investigatory probe, an electrosurgical stimulation device, a coagulation device, an ablation device, a drill, a laser and the like, as is known in the art.

One exemplary tracking sensor 325 for use with the image-guided surgical system 300 is the Optotrak 3020 commercially available from Northern Digital Inc., Waterloo, Ontario, Canada. The optical tracking sensor 325 contains three cylindrical lenses which receive light from sequentially strobed infrared light-emitting diodes (IREDs). Triangulation is used to find each IRED relative to the position of the optical tracking sensor 325. Typical image-guided surgery tracking systems which utilize an optical tracking sensor are described in U.S. Pat. No. 6,584,339 B2.

While the exemplary image-guided surgical system 300 tracks optically using IREDs and an optical tracking sensor technology, other similar tracking technologies can be utilized without departing from the present invention. For example, the image-guided surgical system 300 may use a plurality of cameras (not shown) linked to the computer 305 or control box 315 which detect geometric patterns like the MicronTracker commercially available from Claron Technology, Toronto, Ontario, Canada. Other tracking technologies may include electromagnetic tracking, passive binocular cameras, smart-transmitters/emitters and the like.

In order for the position and orientation of the surgical probe/instrument 320 to be measured by the optical tracking sensor 325, the surgical probe/instrument 320 includes a handle with multiple IREDs distributed over the surface of a handle of the surgical probe/instrument 320 so that at least three IREDs are visible in all of the appropriate orientations of the surgical probe/instrument 320. If three or more IREDs attached to the handle of the surgical probe/instrument 320 are detected by the lenses of the optical tracking sensor 325, the tip of the surgical probe/instrument 320 can be accurately localized in physical space without placing constraints on how the surgical probe/instrument 320 needs to be handled by a user (e.g., a technician, nurse, surgeon or the like). The optical tracking sensor 325 can localize both the surgical probe/instrument 320 and the reference emitter 90 in sensor unit space. By mapping the position of the surgical probe/instrument 320 into the space defined by the position and orientation of the reference emitter 90, the location of the optical tracking sensor 325 drops out of the equations.

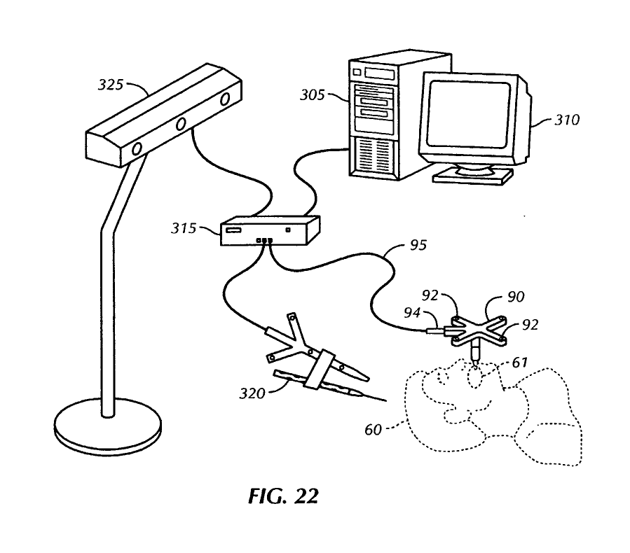

FIG. 22 depicts the exemplary image-guided surgical system 300 in an operating room (OR) environment with the reference emitter 90 attached to the maxillary holding device 50 and a “patient” (shown as a patient's skull 60 in phantom).

FIGS. 4-6 show an “X”-shaped version of the referrence emitter 90. The reference emitter 90 may be other shapes such as a “T”-shaped, a circular-shape and the like without departing from the invention. When the reference emitter 90 is attached directly or, as in the present invention, indirectly to the anatomy, it is possible to allow the anatomy to move relative to the tracking sensor 325 without losing accuracy. It is called a reference emitter 90 because the image-guided surgical system 300 calculates the position of the surgical instrument 320 (e.g., a probe or drill) relative to the reference emitter 90 (i.e., the reference emitter 90 is a point of reference). Thus, when the anatomy moves, it makes no difference because the position of the surgical probe/instrument 320 relative to the anatomy is what is important in an image-guided surgical procedure. Reference emitter IREDs 92 are disposed in or on the reference emitter 90 which are in sync with the image-guidance system 300. The reference emitter 90 includes a connector 94 and cable 95 to electrically couple the reference emitter 90 to the computer of the image-guidance system 300. The IREDs 92 can be “optically” tracked by a position sensor like the optical tracking sensor 325. Because the image-guidance system 300 sequences the IREDs 92 on the reference emitter 90, the image-guidance system 300 can determine the orientation of the reference emitter 90 as monitored by the optical tracking sensor 325.

While the reference emitter 90 is described as being tracked optically using IREDs and an optical tracking sensor technology, other similar tracking technologies can be utilized without departing from the present invention.

Because of possible changes in position or orientation of the reference emitter 90 relative to the fiducial markers 48 on the frame 30, 130, 230, it is desirable, although not necessary, to calibrate the image-guided surgical system 300 more than once. There may be a set of discrete positions, in which case a set of calibrations can be done once for the image-guided surgical system 300. There may also be positions that are not discrete, in which case the calibration must be done for each patient. There may be a combination of these two ideas as well. In particular, a change in relationship between the reference emitter 90 and the location of the fiducial markers 48 installed on the frame 30, 130, 230 is a concern. It is preferred that the reference emitter 90 is not attached to the maxillary holding device 50 during the preoperative image scan because the reference emitter could result in “artifacts” in the image (i.e., potentially deleterious and/or extraneous image information detracting from the imaged anatomy). Therefore, there is a possibility of a chance in position of the reference emitter 90 during installation onto the maxillary holding device 50 after a preoperative scan of the patient and before a surgical procedure.

In one method for performing image-guided surgery using the present invention, the frame 30 with fiducial markers 48 attached is place on the patient without the reference emitter 90 and a preoperative image is acquired using a image scanning system (i.e., CT, MRI, PET, etc.). The frame 30 is then removed from the patient and the reference emitter 90 is attached to the frame 30 and/or maxillary holding device 50, the combination of the frame 30 and/or the maxillary holding device 50 and the reference emitter 90 is then attached to a stationary object for calibration. The frame 30 and maxillary holding device 50 could be left on the patient for calibration, but it would probably be inconvenient to do so. The frame 30 with the fiducial markers 48 is then removed from the maxillary holding device 50 leaving the reference emitter 90 attached directly to the maxillary holding device 50 or requiring the reference emitter 90 to be reattached to the maxillary holding device 50. During a calibration procedure, the fiducial markers 48 may be substituted with fiducial markers 48 having hemispherical divots configured to accept the tip of an optically detectable or MRI detectable surgical probe/instrument 320. The probe is then touched to each of the fiducial markers 48 in order for the image-guided surgical system 300 to calculate the position of the fiducial markers 48 relative to the reference emitter 90. The reference emitter 90 is electrically coupled to the image-guidance system 300 to synchronize the sequencing of the IREDs 92 of the reference emitter 90 and is therefore able to be continuously monitored during surgery. Thus, another significant aspect of the present invention is the ability to use the frame 30 with fiducial markers 48 during a preoperative scan to acquire a patient's “geometry” in image space and to thereafter be able to perform the operation using only the maxillary holding device 50 and reference emitter 90 during surgery. While the calibration step is presently preferred, it is contemplated that the procedure can be accomplished without the calibration step because the size and shape of the frame 30 are known and can be calculated out of the equation without calibration. Calibration is a precautionary measure to compensate for any changes a frame 30 might encounter throughout its re-usable lifetime, changes in the precise installation of a reference emitter 90 and the like. The combination of the maxillary holding device 50 and the reference emitter 90 is then reattached to the patient for use during a surgical procedure.

One application of the present invention is to perform surgery on a patient's ear (i.e., otologic surgery). Otologic surgery, like neurosurgery requires high accuracy, not only for alteration or removal of small structures, but also for the avoidance of nearby structures that must remain untouched. Because the area involved in the procedure is small, it makes sense to increase the accuracy of the image-guidance system 300 in this area, even at the sacrifice of accuracy in remote regions, which will not be involved in the surgery. One fundamental idea of the present invention is to exploit the limited extent of the region in which accuracy must be high, by choosing a marker configuration to provide the maximum accuracy in the region of interest. Furthermore, because the geometric relationship between the mouth and the region of interest will vary according to the nature of the procedure and that patient's particular anatomy, the invention includes the concomitant idea that the choice of configuration will be adjustable. The present invention also includes the idea that the adjustment will be based on a preoperative “estimate” of the effect on the registration accuracy of such an adjusted configuration.

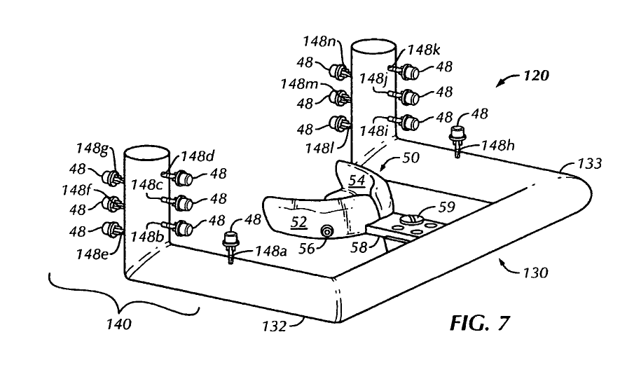

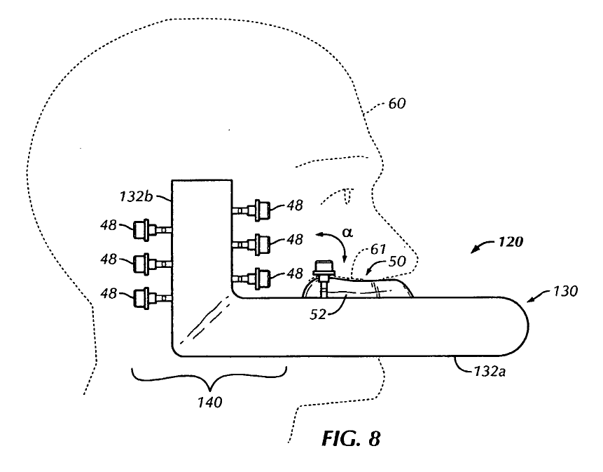

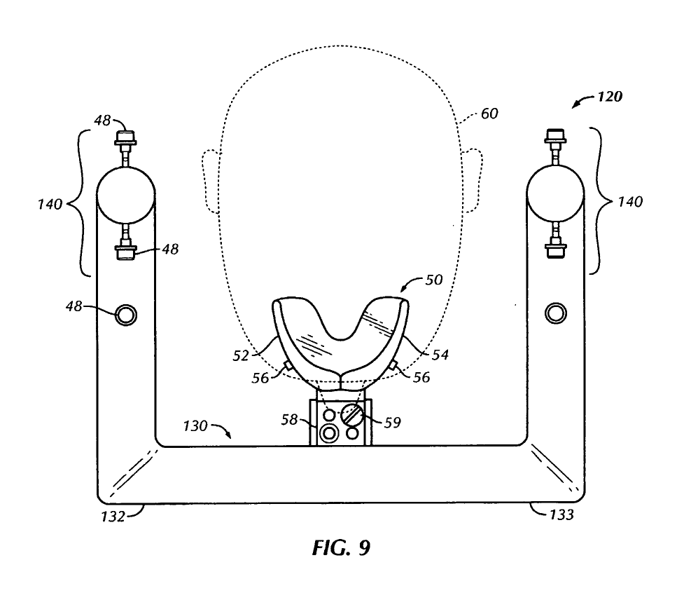

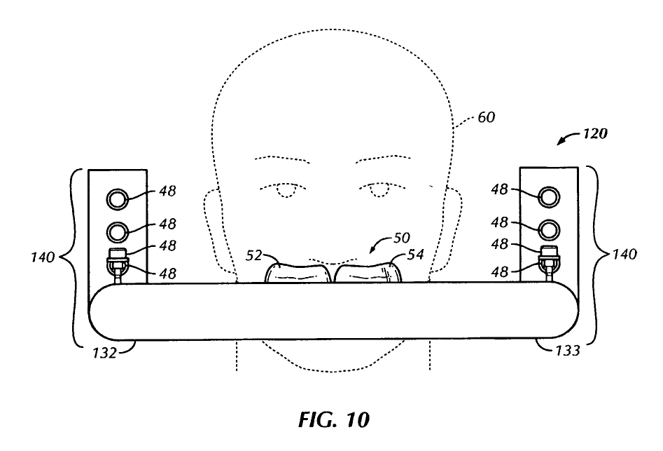

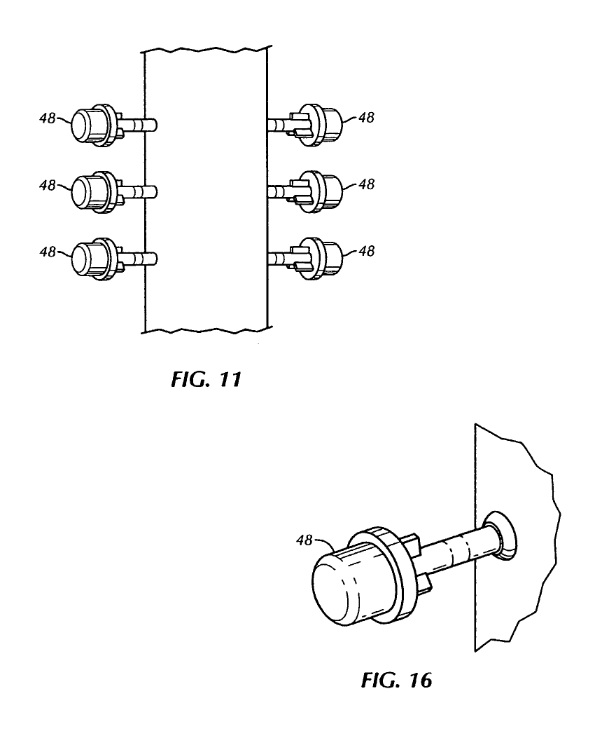

Referring to FIGS. 7-11 there is shown a second preferred embodiment of a fiducial marker holder apparatus for surgery 120 that is shown attached to a maxillary holding device 50 which is attached to the maxillary jaw and/or teeth 61 of a human skull 60. The fiducial marker holder apparatus 120 includes an open-ended frame 130 and marker attachment devices or regions 140. The open-ended frame 130 is configured to be removably attached to the maxillary holding device 50 (shown in detail in FIGS. 4-6 and 19-20). The second preferred embodiment is similar in may aspects to the first preferred embodiment, however, marker attachment devices 140 are integrally formed with the frame 130 in the shape of a cylinder providing another unique way to attach fiducial markers 48 in a concentrated area or region with improved resolution for a particular image-guided surgical procedure and the like. The marker attachment devices 140 are disposed on a first end or arm 132 and a second end or arm 133 of the open-ended frame 130, respectively. The marker attachment devices 140 include a plurality of marker attachment points 148a-148n. Preferably, the first arm 132 has at least one marker attachment point 148a-148n that receives fiducial markers 48 and the second arm 133 has a plurality of marker attachment points 148a-148n that receive fiducial markers 48, and at least two of the marker attachment points 148a-148n of the second arm 133 are configured to receive fiducial markers 48 in different orientations with respect to the open-ended frame 130 and each other 148a-148n. Each marker attachment device 140 has multiple, distinct geometric surfaces and the marker attachment points 148a-148n are disposed on two or more of the surfaces of the marker attachment device 140 thereby facilitating arranging the fiducial markers 48 in different orientations with respect to the open-ended frame 130 and some other fiducial markers 48. Thus, fiducial markers 48 on each of the arms 132, 133 can be arranged in two or more dimensions with respect to the arm 132, 133 to which they are attached. For example, marker attachment point 148b is directed forward or in a direction toward the front of the patient, marker attachment point 148g is directed rearward or in a direction toward the back of the patient, and marker attachment point 148a is directed upward or in a direction toward the top of a patient's head. Of course varying directions are also possible at a plurality of angles over the surface of the arms 132, 133. As shown, the first arm 132 has a first portion 132a and a second portion 132b extending at an angle α from the first portion 132, and the second arm 133 has a first portion 133a and a second portion 133b extending at an angle α from the first portion 133a. Additionally, different shapes of the arms 132, 133, in lieu of the L-shape depicted in FIG. 7, could also effectively change the orientation of the various marker attachment points 148a-148n with respect to the open-ended frame 130 and each other 148a-148n. FIG. 11 is a greatly enlarged view of the fiducial marker attachment device 140.

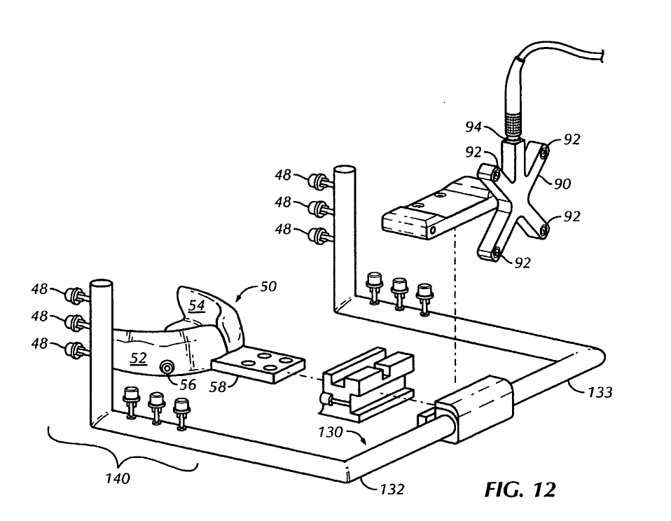

Referring to FIGS. 12, 13A-13C, 14A-14C and 15A-15C, there is shown a third preferred embodiment of a fiducial marker holder apparatus for surgery 220 that is shown attached to a maxillary holding device 50 which is attached to the maxillary jaw and/or teeth 61 of a human skull 60. The fiducial marker holder apparatus 220 includes an open-ended frame 230 and a marker attachment device 240. The open-ended frame 230 is configured to be removably attached to the maxillary holding device 50. The second preferred embodiment is similar in may aspects to the first preferred embodiment, however, marker attachment devices 240 are integrally formed with the open-ended frame 230 in an L-shaped cylindrical structure providing another unique way to attach fiducial markers 48 in a concentrated area or region with improved resolution for a particular image-guided surgical procedure and the like. The fiducial marker attachment devices 240 may be other shapes and configurations such as curvilinear, square, circular, X-shaped and the like without departing from the invention. The marker attachment devices 240 are disposed at a first end or arm 232 and a second end or arm 233 of the open-ended frame 230, respectively. The marker attachment devices 240 include a plurality of marker attachment points 248a-248l. Preferably, the first arm 232 has at least one marker attachment point 248a-248l that receives fiducial markers 48 and the second arm 233 has a plurality of marker attachment points 248a-248l that receive fiducial markers 48, and at least two of the marker attachment points 248a-248l of the second arm 233 are configured to receive fiducial markers 48 in different orientations with respect to the open-ended frame 230 and each other 248a-248l. Each marker attachment device 240 has multiple, distinct geometric surfaces and the marker attachment points 248a-248l are disposed on two or more of the surfaces of the marker attachment device 240 thereby facilitating arranging the fiducial markers 48 in different orientations with respect to the open-ended frame 230 and some other fiducial markers 48. Thus, fiducial markers 48 on each of the arms 232, 233 can be arranged in two or more dimensions with respect to the arm 232, 233 to which they are attached. Thus, fiducial markers 48 on each of the arms 232, 233 can be arranged in two or more dimensions with respect to the arm 232, 233 to which they are attached. For example, marker attachment point 248d is directed rearward or in a direction toward the back of the patient and marker attachment point 248a is directed upward or in a direction toward the top of a patient's head. Of course varying directions are also possible at a plurality of angles over the surface of the arms 232, 233. As shown, the first arm 232 has a first portion 232a and a second portion 232b extending at an angle α from the first portion 232, and the second arm 233 has a first portion 233a and a second portion 233b extending at an angle α from the first portion 233a. Additionally, different shapes of the arms 232, 233, in lieu of the L-shape depicted in FIGS. 12, 13A-13C and 14A-14C, could also effectively change the orientation of the various marker attachment points 248a-248l with respect to the open-ended frame 230 and each other 248a-248l.

FIG. 12 is a the fiducial marker holder apparatus for surgery 220 before being attached to the maxillary holding device 50 which will be attached to the maxillary jaw and/or teeth 61 of a human skull 60 along with the reference emitter 90.

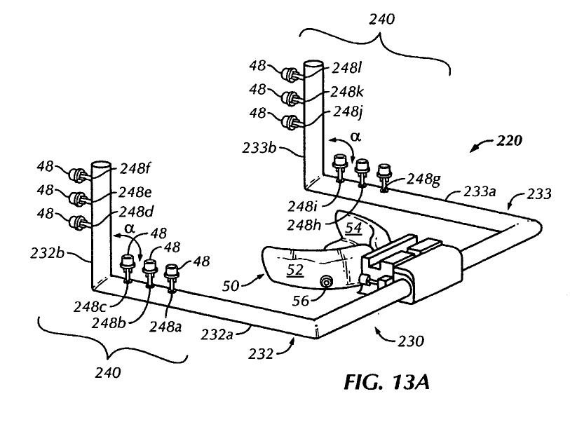

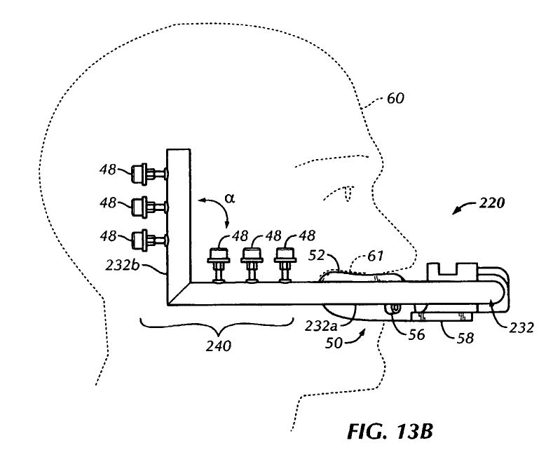

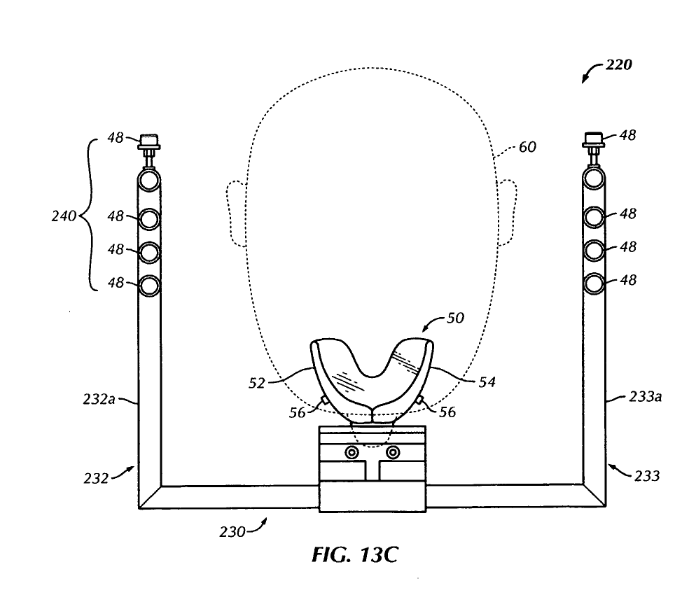

FIGS. 13A-13B show the fiducial marker holder apparatus 220 configured for a preoperative scan.

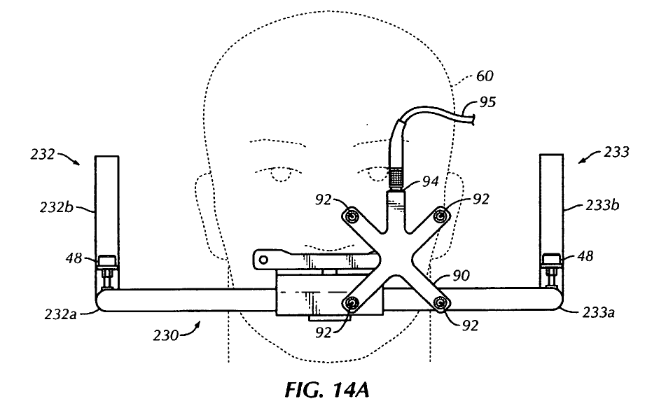

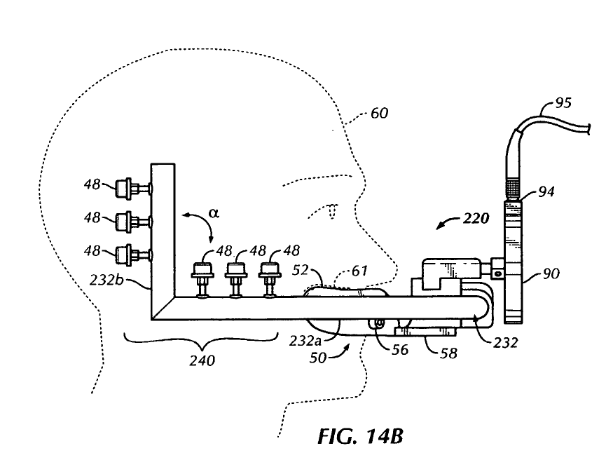

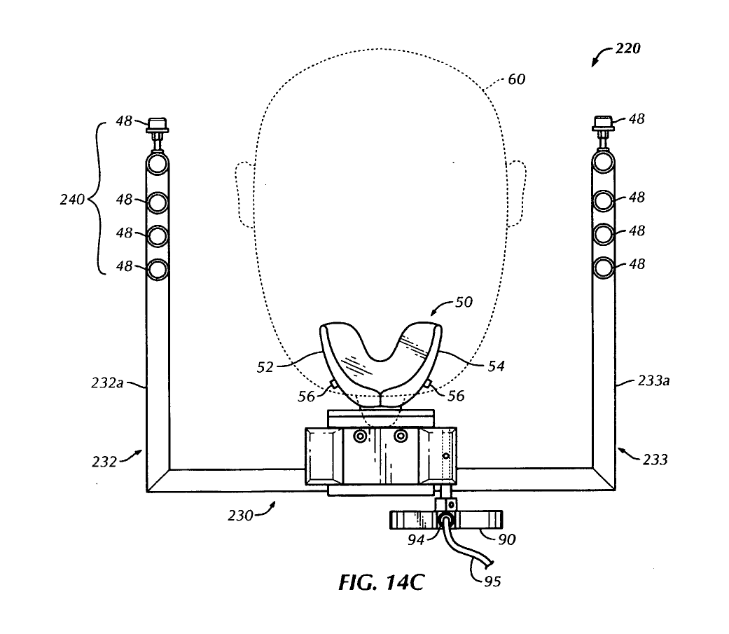

FIGS. 14A-14C show the fiducial marker holder apparatus 220 and maxillary holding device 50 with the reference emitter 90 attached and configured for a calibration procedure.

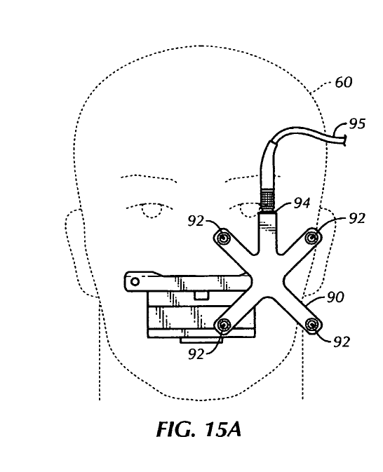

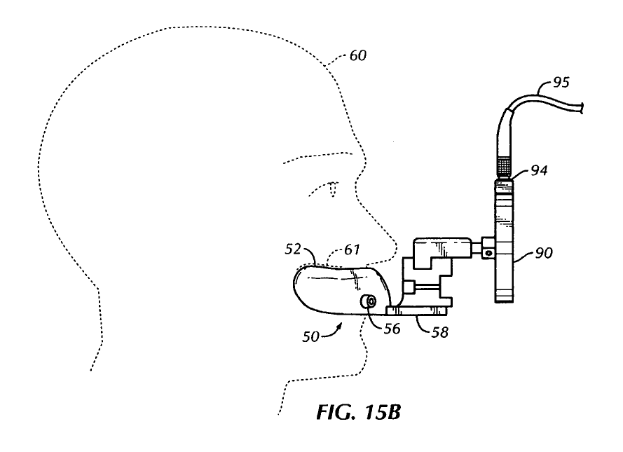

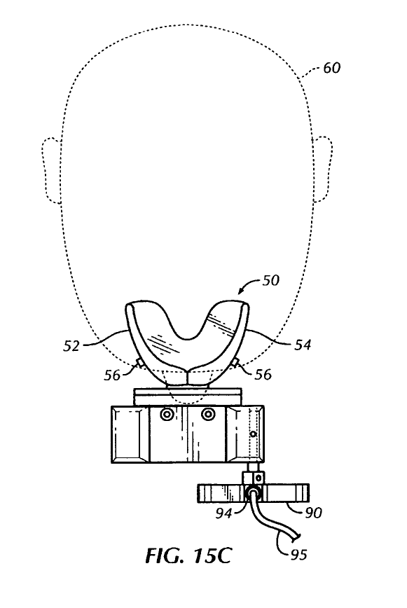

FIGS. 15A-15C show the maxillary holding device 50 with the reference emitter 90 attached and configured for a surgical procedure.

Another method for performing image-guided surgery using the present invention, includes using any of the preferred embodiments of the present invention in conjunction with the surgical probe/instrument 320, the reference emitter 90 and the image-guided surgical system 300 as follows:

(i) attaching the frame 30, 130, 230 with fiducial markers 48 to a patient and a acquiring a preoperative scanned image (see for e.g., FIGS. 13A-13C);

(ii) removing the frame 30, 130, 230 from the patient;

(iii) making a surgical plan, by the surgeon, from the preoperative scanned image;

(iv) calculating the position of any point in or on the patient relative to the frame 30, 130, 230;

(v) attaching the reference emitter 90 to the frame 30, 130, 230 (see for e.g., FIGS. 14A-14C);

(vi) activating the image-guided surgical system 300 which then begins tracking the reference emitter 90;

(vii) calibrating the frame 30, 130, 230 and the reference emitter 90, while the patient is being prepared for surgery, so that the position of the frame 30, 130, 230 relative to the reference emitter 90 is determined by the image-guided surgical system 300;

(viii) continuously calculating the position of the frame 30, 130, 230 relative to the reference emitter 90, no matter how the frame 30, 130, 230 and the reference emitter 90 move;

(ix) attaching the maxillary holding device 50 with the attached reference emitter 90 to the patient without the frame 30, 130, 230 (see for e.g., FIGS. 15A-15C);

(x) calculating the position of any point in the imaged-patient anatomy relative to the reference emitter 90;

(xi) activating the surgical probe/instrument 320;

(xii) tracking the reference emitter 90 and the surgical probe/instrument 320 simultaneously;

(xiii) calculating the position of the surgical instrument relative to the patient's anatomy; and

(xiv) using the image-guided surgical system 300 to guide surgery.

Thus, surgical intervention (i.e., the actual surgery or surgical procedure) proceeds after imaging studies or scans are obtained (i.e., CT scans and the like) which can be used to prepare for surgery and which are referenced during surgery. During surgery, which is performed using an operating microscope and surgical drill for example, visual feedback and surgical experience are the safeguards used to ensure accurate dissection Likewise, the image-guided surgical system 300 that is used to perform image-guided surgery may be calibrated using a procedure such as the following:

(i) attaching the open-ended frame 30, 130, 230 with the plurality of fiducial markers 48 to a patient using the maxillary holding device 50;

(ii) acquiring a preoperative scan of the patient and the open-ended frame 30, 130, 230 with the plurality of fiducial markers 48;

(iii) removing the maxillary holding device 50 from the patient;

(iv) attaching the reference emitter 90 to either the open-ended frame 30, 130, 230 or the maxillary holding device 50;

(v) activating the tracking sensor 325 which then begins tracking the reference emitter 90;

(vi) activating the surgical probe/instrument 320;

(vii) tracking the reference emitter 90 and the surgical probe/instrument 320 simultaneously and continuously calculating the position of the surgical probe/instrument 320 relative to the reference emitter 90; and

(viii) calibrating the image-guided surgical system 300 with respect to the frame 30, 130, 230 and the reference emitter 90 by touching the surgical probe/instrument 320 to each fiducial marker 48, so that the position of the frame 30, 130, 230 relative to the reference emitter 90 is determined and stored in the memory of the image-guided surgical system 300.

Once a calibration procedure has been performed, the image-guided surgical system 300 can be used to perform surgery with or without the frame 30, 130, 230 because the patient's anatomy can be tracked relative to the reference emitter 90.

General Image Registration:

The general concept is to use information from radiographic images to provide guidance to the surgeon thereby reducing problems associated with comparing three-dimensional positional measurements in the operating room (OR) with corresponding measurements in pre-operative images. This comparison requires that points in the imaged anatomy be mapped onto points in the actual anatomy by image registration Image registration in human anatomy is, in general, a complex mathematical problem because organs are deformable (i.e., lack rigidity). In otologic surgery, however, the “organ” of interest (the ear) is housed in a rigid body (the temporal bone), and therefore, the ear can be treated, for all practical purposes, as a rigid body. Image registration for rigid bodies, while still complex, is much simpler than image registration for deformable bodies.

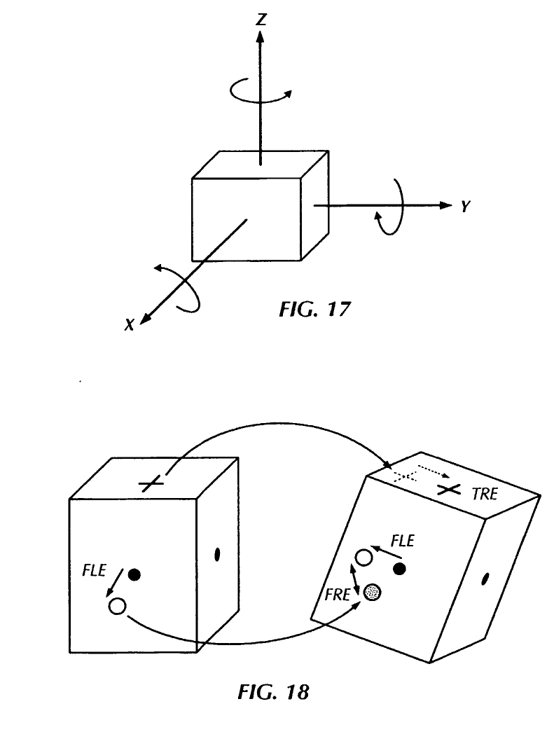

To determine the mapping from a rigid body to its image only a few parameters need be determined. They are the shifts in x, y, and z and the angular rotations about each of these three axes, as shown in FIG. 17. The anatomy in each discrete voxel (volume element) in the image moves as a simple and predictable function of these six parameters. A voxel is the smallest distinguishable box-shaped part of a three-dimensional space, and a particular voxel can be identified by the x, y and z coordinates of one of its eight corners or its center. Real world applications, however, are not that simple, because of unavoidable error, called fiducial localization error (FLE) which occurs when identifying the fiducial markers. Thus, image registration must also account for this error. Minimizing the overall registration error and tracking it through the registration process is the difficult part of rigid-body registration.

An approach for reducing registration error, when there are errors in the point measurements, is to use more than the minimum of three points. While there will almost never be a rigid transformation that will align them exactly, it can be expected that a transformation that fits them as closely as possible is the best one. As with most fitting problems involving measurement error the optimal fit is the least-squares fit, which minimizes the sum of the squares of the distances between the corresponding points. The square root of this sum of squares is called the fiducial registration error (FRE) because it gives a single measure in millimeters of how well the fiducials are brought into registration. Minimizing FRE has been found to be the best approach. From this solution, described below, FRE is calculated and displayed in order to provide feedback to the surgeon with regard to the accuracy of the surgical guidance.

The two types of error, FLE and FRE, are important to the understanding of the problem of IGS, but neither of them is the error of importance to a surgeon. The important error to a surgeon is the error incurred during identification of surgical targets. These errors are termed target registration errors (TRE) and they provide another example of a difficult mathematical problem associated with point-based registration which is the calculation of the effect of the input error (FLE) oil the output error (TRE). The ability to calculate the effect of the input error (FLE) on the output error (TRE) has made it possible to design systems that can minimize output error (TRE) and has made it possible to estimate the level of output error (TRE) that can be expected for a given system with a given level of input error (FLE).

FIG. 18 shows an image of a box (left) that has been registered to itself (right) using fiducial markers 48 located on its sides. The solid black dots are the true centroids of the fiducial markers 48. The open circles are the localized centroids (shown only for the front centroid). The single-headed arrows show the FLE in each space for this marker. The shaded circle is the position to which this marker's localized position is mapped by the registration. The solid double-headed arrow shows the FRE for this marker. The solid plus signs at the top of the box are the true positions of a target point in each space. The dotted plus sign represents the position to which the image target point is mapped by the registration. Thus, the dotted single-headed arrow shows TRE of the target.

For application to otologic surgery, repeatable registration errors of 1 millimeter (mm) and less are crucial as the surgical anatomy of the temporal bone presents with diseased tissue in close proximity to vital tissue. In addition to registration error, measurement error exists (e.g. secondary to the finite pixel size of the image, the noise in image intensity measurements, the uncertainty in the intraoperative tracking of the surgical probe/instrument 320, imprecision in the positioning of fiducial markers 48 relative to the maxillary holding device 50, small movements of the maxillary holding device 50 relative to the skull, etc.) which in similar applications have been shown to be approximately 1 mm. As the measurement error approaches the approximate magnitude of the registration error, careful error analysis must be performed during the development of the hardware and the software that make up the guidance system to control the error propagation through the system.

Image Registration Analysis:

One registration method in accordance with the preferred embodiments of the present invention includes determining a rotation matrix and translation vector that provide a rigid-body transformation which minimizes the sum of squares of the distances between corresponding points in two spaces. Once the centroid points of Nₚ markers have been localized in each space, there are 2Nₚ vectors as inputs that give the coordinates of the points in the respective spaces,
p₁, . . . , p_{N}_{ᵣ}
q₁, . . . , q_{N}_{ᵣ} (1)
where pᵢ represents a point in image space and qᵢ represents a point in anatomic space. The outputs are one rotation matrix R and one translation vector t. The transformation then has the form:
pᵢ′=Rpᵢ+t, (2)
for i=1, . . . , Nₚ. While any of the closed-form solutions may be employed, an explanation is provided here using the Singular-Value-Decomposition method. The translation is given by the displacement between the weighted means of the two point sets:
t=\overline{q}−\overline{p}, (3)
where

$$H = {\sum }_{i = 1}^{{N}_{p}} {w}_{i}^{( p )} {\overline{p}}_{i} {\overline{q}}_{i}^{′} \qquad ( 5 )$$

Then, a cross-covariance matrix is formed,

$$\overline{p} = \sum {w}_{i}^{( p )} {p}_{i} / \sum {w}_{i}^{( p )} , \overline{q} = \sum {w}_{i}^{( p )} {q}_{i} / \sum {w}_{i}^{( p )} . \qquad ( 4 )$$

where
\tilde{p}ᵢ=pᵢ−\overline{p}ᵢ, \tilde{q}ᵢ=qᵢ−\overline{q}ᵢ, (6)

The wᵢ^{(p)} are weights which may be adjusted according to the certainty of the measurements. Singular value decomposition of H is effected: UAV′=H.

Finally,
R=U′diag(1,1,det(UV))V, (7)
where “diag” is a diagonal matrix with the indicated elements on the diagonal and “det” means “determinant of”.

Once the registration is effected, it is of interest to measure the fiducial registration error (FRE) and the target registration error (TRE). Both of these error measures are dependent on the fiducial localization error (FLE). FRE is defined as follows:

$${FRE}^{2} = \frac{1}{{N}_{p}} {\sum }_{i = 1}^{{N}_{p}} {\vert {p}_{i}^{′} - {q}_{i} \vert}^{2} \qquad ( 8 )$$

As can be seen from its definition FRE is a measure of the goodness of fit of the marker centroids after registration. This statistic can be predicted on the basis of FLE. The relationship was first given by Sibson, as follows: [symbol]
FRE² [symbol] =(1−2/Nₚ) [symbol] FLE² [symbol] (9)
where “ [symbol] . [symbol] ” indicates expected value. TRE is defined as follows:
TRE²=|Rp^{(t)}+t−q^{(t)}|² (10)
where p^{(t)} is the position of the target in image space and q^{(t)} is the corresponding position in anatomic space. Thus, TRE measures the distance between the registered image position and the anatomic position. This statistic can be predicted on the basis of FLE and the positions of the markers and the target position. After the principal axes of the marker configuration is found, the expression is given as follows:

$$〈 {TRE}^{2} 〉 = \frac{1}{{N}_{p}} ( 1 + \frac{1}{3} {\sum }_{k = t}^{3} \frac{{d}_{k}^{2}}{{f}_{k}^{2}} ) 〈 {FLE}^{2} 〉 , \qquad ( 11 )$$

where dₖ is the distance of the target from principal axis k, and fₖ is the root-mean square distance of the marker centroids from the same axis.

Registration accuracy can be determined by using Eq. (10). The positions of a set of easily localized anatomic targets are measured in both the image space p^{(t)} and image space q^{(t)}. Then, for each target in turn, these two positions are used along with the R and t as determined from Eqs. (7) and (3) in Eq. (10) to determine TRE for the given target. Eq. (11) need not be used directly for TRE measurements. Instead, it can serve as an indicator and as a check on the calculated results. In order to use Eq. (11), the expected value of FLE must be known or assumed. Such information can be determined by measuring FRE for sets of registrations, calculating the mean value of FRE² as an estimate of [symbol] FRE² [symbol] , and then solving Eq. (9) for FLE.

Dental Splints

From the foregoing, it can be seen that the present invention comprises a fiducial marker holder system for surgery adapted to increase registration accuracy, especially during otologic surgical procedures and that the present invention also provides a fiducial marker holder system and image-guided otologic surgical system combination having accuracies in the submillimeter range and which is non-invasive and non-obstructive. It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A fiducial marker holder apparatus for image-guided surgery comprising:
an open-ended frame having first and second arms, the open-ended frame being configured to be removably attached to a maxillary holding device and the first arm having at least one marker attachment point that receives fiducial markers; and
a marker attachment device disposed at a distal end of the second arm of the open-ended frame, the marker attachment device having a plurality of marker attachment points that receive fiducial markers, at least two of the marker attachment points of the marker attachment device being configured to receive fiducial markers in different orientations with respect to the marker attachment device and each other.

2. The fiducial marker holder apparatus according to claim 1, wherein the marker attachment device is movably attached to the second arm of the open-ended frame.

3. The fiducial marker holder apparatus according to claim 2, wherein the marker attachment device is configured to be fixedly oriented in more than one position.

4. The fiducial marker holder apparatus according to claim 1, further comprising an additional marker attachment device disposed at a distal end of the first arm of the open-ended frame, the additional marker attachment device having a plurality of marker attachment points that receive fiducial markers.

5. The fiducial marker holder apparatus according to claim 1, wherein the marker attachment points are each configured as one of a threaded socket, a threaded post, a through-hole, a post, a socket and a detent.

6. A fiducial marker holder apparatus for image-guided surgery comprising:
a maxillary holding device configured to be temporarily secured to only a maxillary-region of a patient;
an open-ended frame having first and second arms, the open-ended frame being configured to be removably attached to the maxillary holding device, the first arm having at least one marker attachment point that receives fiducial markers and the second arm having a plurality of marker attachment points that receive fiducial markers, at least two of the marker attachment points of the second arm being configured to receive fiducial markers in different orientations with respect to the open-ended frame and each other.

7. The fiducial marker holder apparatus according to claim 6, wherein the open-ended frame is rigid.

8. The fiducial marker holder apparatus according to claim 6, wherein the open-ended frame is formed of one of a carbon-fiber material, a non-metallic composite, and a polymeric material.

9. The fiducial marker holder apparatus according to claim 6, wherein the marker attachment points are each configured as one of a threaded socket, a threaded post, a through-hole, a post, a socket and a detent.

10. The fiducial marker holder apparatus according to claim 6, wherein the second arm has a first portion and a second portion extending at an angle from the first portion.

11. The fiducial marker holder apparatus according to claim 6, wherein the first arm has a plurality of marker attachment points that receive fiducial markers and the first arm has a first portion and a second portion extending at an angle from the first portion.

12. The fiducial marker holder apparatus according to claim 6, wherein the maxillary holding device includes a locking dental acrylic resin splint that is custom molded for a particular patient.

13. The fiducial marker holder apparatus according to claim 6, wherein the maxillary holding device is fastened directly to the patient by fasteners.

14. A fiducial marker holder system for image-guided surgery comprising:
a maxillary holding device having a first clamping part, a second clamping part and a fixing tool, the fixing tool being configured to temporarily secure the first and second clamping parts to only a maxillary-region of a patient;
an open-ended frame having first and second arms, the open-ended frame being configured to be removably attached to the maxillary holding device, the first arm having at least one marker attachment point that receives fiducial markers and the second arm having a plurality of marker attachment points that receive fiducial markers, at least two of the marker attachment points of the second arm being configured to receive fiducial markers in different orientations with respect to the open-ended frame and each other; and
a reference emitter configured to be removably attached to the maxillary holding device or the open-ended frame.

15. The fiducial marker holder system according to claim 14, wherein the open-ended frame is formed of a rigid material.

16. The fiducial marker holder system according to claim 14, wherein the first arm has a plurality of marker attachment points that receive fiducial markers.

17. The fiducial marker holder apparatus according to claim 14, wherein the marker attachment points are each configured as one of a threaded socket, a threaded post, a through-hole, a post, a socket and a detent.

18. A method of performing image-guided surgery on a patient using a maxillary holding device, an open-ended frame, a plurality of fiducial markers, a reference emitter, a surgical probe/instrument and an image-guided surgical system having a tracking sensor, the method comprising:
(a) attaching the open-ended frame with the plurality of fiducial markers to a patient using the maxillary holding device;
(b) acquiring a preoperative scan of the patient and the open-ended frame with the plurality of fiducial markers;
(c) removing the maxillary holding device from the patient;
(d) making a surgical plan, by the surgeon, from the preoperative scan;
(e) calculating the position of any point in or on the patient relative to the frame;
(f) attaching the reference emitter to the frame;
(g) activating the tracking sensor which then begins tracking the reference emitter and the frame;
(h) calibrating the frame and the reference emitter, while the patient is being prepared for surgery, so that the position of the frame relative to the reference emitter is determined;
(i) calculating the position of the frame relative to the reference emitter;
(j) removing the frame with the plurality of fiducial markers from maxillary holding device;
(k) attaching the maxillary holding device with the reference emitter to the patient;
(l) calculating the position of any point in the intraoperative-imaged patient anatomy relative to the reference emitter;
(m) activating a surgical probe/instrument;
(n) tracking the reference emitter and the surgical probe/instrument simultaneously;
(o) calculating the position of the surgical probe/instrument relative to the patient's anatomy; and
(p) using the image-guided surgical system to guide surgery.

19. A method of calibrating an image-guided surgical system that is used to perform image-guided surgery on a patient using a maxillary holding device, an open-ended frame, a plurality of fiducial markers, a reference emitter, a surgical probe/instrument and the image-guided surgical system having memory and a tracking sensor, the method comprising:
(a) attaching the open-ended frame with the plurality of fiducial markers to a patient using the maxillary holding device;
(b) acquiring a preoperative scan of the patient and the open-ended frame with the plurality of fiducial markers;
(c) removing the maxillary holding device from the patient;
(d) attaching the reference emitter to either the open-ended frame or the maxillary holding device;
(e) activating the tracking sensor which then begins tracking the reference emitter;
(f) activating the surgical probe/instrument;
(g) tracking the reference emitter and the surgical probe/instrument simultaneously and continuously calculating the position of the surgical probe/instrument relative to the reference emitter; and
(h) calibrating the image-guided surgical system with respect to the frame and the reference emitter by touching the surgical probe/instrument to each fiducial marker, so that the position of the frame relative to the reference emitter is determined and stored in the memory of the image-guided surgical system.
